(19) United States
(12) Patent Application Publication
(10) Pub. No.: US 2025/0273373 A1
(43) Pub. Date: Aug. 28, 2025

(54) SUPPORT DEVICE FOR MAGNETIC FIELD GENERATOR

(71) Applicant: PLA GENERAL HOSPITAL OF NORTHERN THEATER COMMAND, Shenyang City (CN)

(72) Inventors: Huishan WANG, Shenyang City (CN); Yuji Zhang, Shenyang City (CN); Zongtao YIN, Shenyang City (CN)

(21) Appl. No.: 18/823,044

(22) Filed: Sep. 3, 2024

(30) Foreign Application Priority Data
Feb. 28, 2024 (CN) 202410226589.0

(51) Int. Cl. H01F 7/02; G01D 5/14

(52) U.S. Cl. CPC H01F 7/0294; G01D 5/145

(57) ABSTRACT

Provided is a support device for a magnetic field generator, relating to the technical field of medical devices. The support device includes a cardiac surgery bed, and further includes a support column arranged at the bottom of the cardiac surgery bed. An output end of a servo motor is driven to make a first threaded rod rotate, the rotation of the first threaded rod drives a first driving wheel to rotate, the rotation of the first driving wheel drives a conveyor belt to drive, then the conveyor belt drives a second driving wheel to rotate to make a second threaded rod to rotate, thus making a thread bushing move. When the thread bushing moves, an extension plate is driven to move, and when the extension plate moves, a limiting shell is driven to move, thus adjusting a position of a mapping assembly.

### CROSS-REFERENCE TO RELATED APPLICATION

This patent application claims the benefit and priority of Chinese Patent Application No. 202410226589.0, filed with the China National Intellectual Property Administration on Feb. 28, 2024, the disclosure of which is incorporated by reference herein in its entirety as part of the present application.

### TECHNICAL FIELD

The present disclosure relates to the technical field of medical devices, and in particular to a support device for a magnetic field generator.

### BACKGROUND

Magnetic field generator is a constituent part of electrophysiological mapping, which receives two signals in total, one is a magnetic field signal and the other is an electric signal. The magnetic field signal is realized by a magnetic field generator, which can play a positioning role. The electric signal is realized by an electrode, and the electrical signal at this site is recorded.

In surgery, cardiac electrophysiological mapping can predict the preoperative state of the patient and evaluate the surgical effect, so as to improve the surgical method. However, because there is no specially designed magnetic field generator mounting bracket on the cardiac surgical bed and no magnetic field generator mounting bracket that can be directly installed on the cardiac surgical table, it is difficult and costly to directly mount a magnetic field generator on the surgical bed.

### SUMMARY

An objective of the present disclosure is to provide a support device for a magnetic field generator, so to solve the problems in the background above.

To achieve the objective, the present disclosure provides the following technical solutions: a support device for a magnetic field generator includes a cardiac surgery bed, and further includes: a support column arranged at the bottom of the cardiac surgery bed, where a connecting support shell is arranged on a surface of the cardiac surgery bed, connecting plates are fixedly connected to both sides of the bottom of the connecting support shell, and the top of each connecting plate is fixedly connected to the cardiac surgery bed; a moving assembly arranged on one side of the connecting support shell, where a mapping assembly is arranged at the top of the cardiac surgery bed, and provided with mounting plates at four corners of the bottom; and a guide plate arranged at the bottom of each mounting plate, where a positioning shell is arranged on a surface of the guide plate, and an adjusting assembly is arranged in an inner cavity of the positioning shell.

Preferably, a rib-reinforced plate is fixedly connected to one side of the connecting plate, and the top of the rib-reinforced plate is fixedly connected to the cardiac surgery bed.

Preferably, the moving assembly includes a servo motor. A left side of the servo motor is fixedly connected to the connecting support shell, and an output end of the servo motor runs through an inner cavity of the connecting support shell and is fixedly connected with a first threaded rod. A first driving wheel is fixedly connected to one side of a surface of the first threaded rod, a surface of the first driving wheel is sleeved with a conveyor belt, a front side of an inner cavity of the conveyor belt is in transmission connection with a second driving wheel, and a second threaded rod is fixedly connected to an inner cavity of the second driving wheel.

Preferably, both sides of the surface of each of the first threaded rod and the second threaded rod are sleeved with thread bushings. An extension plate is fixedly connected to the top of each thread bushing, and a limiting shell is fixedly connected to the top of the extension plate.

Preferably, both sides of each of the first threaded rod and the second threaded rod are provided with bearing housings, and opposite sides of the two bearing housings are both fixedly connected to an inner wall of the connecting support shell.

Preferably, the mapping assembly includes a protective shell. Mounting holes are formed in four corners of the top of the protective shell, and a hardware assembly is fixedly connected to the top of the protective shell.

Preferably, the protective shell is made of PA (polyamide), and the hardware assembly includes a magnetic field generator.

Preferably, the adjusting assembly includes a spring set. A front side of the spring set is fixedly connected to an inner wall of the positioning shell, a positioning plate is fixedly connected to one side of the spring set, a connecting rod is fixedly connected to one side of the positioning plate, and one side of the connecting rod runs through to the outside of the positioning shell and is fixedly connected with a circular pull ring. A tooth block is fixedly connected to one side of the positioning plate, and an opening in cooperative use with the tooth block is formed in one side of the guide plate.

Preferably, sliders are fixedly connected to both sides of the positioning plate, and opposite sides of the two sliders both run through to the outside of the positioning shell. Opening holes in cooperative use with the sliders are formed in both sides of the top and bottom of the inner cavity of the positioning shell.

Preferably, a surface of the connecting rod is sleeved with a retaining ring, and one side, close to the positioning shell, of the retaining ring is fixedly connected to the positioning shell.

Compared with the prior art, the present disclosure has beneficial effects as follows:

When a user needs to adjust the mapping assembly to move left and right, an output end of the servo motor is driven to make the first threaded rod rotate, the rotation of the first threaded rod drives the first driving wheel to rotate, the rotation of the first driving wheel drives the conveyor belt to drive, then the conveyor belt drives the second driving wheel to rotate to make the second threaded rod rotate. In this way, the first threaded rod and the second threaded rod both rotate in the inner cavity of the bearing housing, making the thread bushing move. When the thread bushing moves, the extension plate is driven to move, and when the extension plate moves, the limiting shell is driven to move, thus adjusting a position of the mapping assembly. When the user needs to adjust the height of the mapping assembly, the circular pull ring can be pulled to make the connecting rod move in an inner cavity of the retaining ring in a limited manner, when the connecting rod moves, the positioning plate is driven to move, when the positioning plate moves, the slider is driven to move in a limited manner to extrude the spring seat, and when the positioning plate moves, the tooth block is driven to be separated from the opening on the guide plate. In this way, the guide plate can be dragged to drive the mounting plate and the mapping assembly to ascend or descend, then the circular pull ring is released, and the resetting of the spring set enables the positioning plate to drive the tooth block to be inserted into the opening on the guide plate, thus fixing the adjusted height. The protective shell is convenient for the mounting of the hardware assembly, can effectively protect the mapping assembly due to its high temperature resistance and corrosion resistance. The protective shell is made of PA, which can be disinfected at a high temperature of 120° C., and is more hygienic and safer.

### BRIEF DESCRIPTION OF THE DRAWINGS

FIG. 1 is a structural diagram of a preferred embodiment of a support device for a magnetic field generator according to the present disclosure;

FIG. 2 is a schematic diagram of a top structure of a cardiac surgery bed according to the present disclosure;

FIG. 3 is a structural diagram of a moving assembly according to the present disclosure;

FIG. 4 is a structural diagram of a mapping assembly according to the present disclosure;

FIG. 5 is a structural diagram of an adjusting assembly according to the present disclosure.

In the drawings: 1—cardiac surgery bed; 2—support column; 3—connecting support shell; 4—connecting plate; 5—moving assembly; 501—servo motor; 502—first threaded rod; 503—first driving wheel; 504—conveyor belt; 505—second driving wheel; 506—second threaded rod; 507—thread bushing; 508—extension plate; 509—limiting shell; 510—bearing housing; 6—mapping assembly; 601—protective shell; 602—mounting hole; 603—hardware assembly; 7—mounting plate; 8—guide plate; 9—positioning shell; 10—adjusting assembly; 101—spring set; 102—positioning plate; 103—connecting rod; 104—circular pull ring; 105—tooth block; 106—slider; 107—retaining ring; 11—rib-reinforced plate.

### DETAILED DESCRIPTION OF THE EMBODIMENTS

The following clearly and completely describes the technical solutions in the embodiments of the present disclosure with reference to the accompanying drawings in the embodiments of the present disclosure. Apparently, the described embodiments are merely a part rather than all of the embodiments of the present disclosure. All other embodiments obtained by those of ordinary skill in the art based on the embodiments of the present disclosure without creative efforts shall fall within the protection scope of the present disclosure.

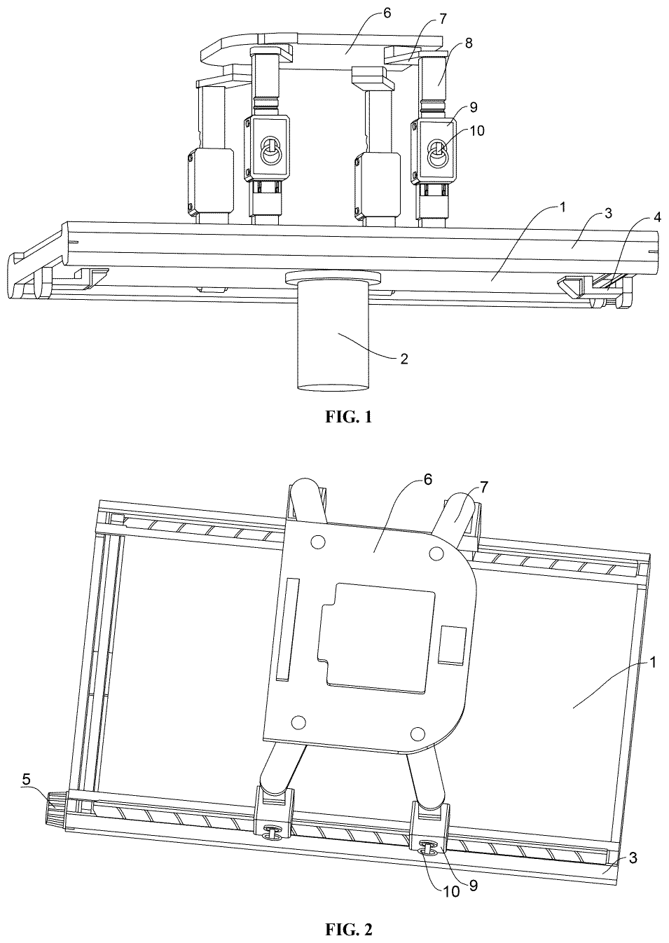

As shown in FIG. 1 to FIG. 5, a support device for a magnetic field generator includes a cardiac surgery bed 1, and further includes: a support column 2 arranged at the bottom of the cardiac surgery bed 1, where a connecting support shell 3 is arranged on a surface of the cardiac surgery bed 1, connecting plates 4 are fixedly connected to both sides of the bottom of the connecting support shell 3, and the top of each connecting plate 4 is fixedly connected to the cardiac surgery bed 1; a moving assembly 5 arranged on one side of the connecting support shell 3, where a mapping assembly 6 is arranged at the top of the cardiac surgery bed 1, and provided with mounting plates 7 at four corners of the bottom; and a guide plate 8 arranged at the bottom of each mounting plate 7, where a positioning shell 9 is arranged on a surface of the guide plate 8, and an adjusting assembly 10 is arranged in an inner cavity of the positioning shell 9.

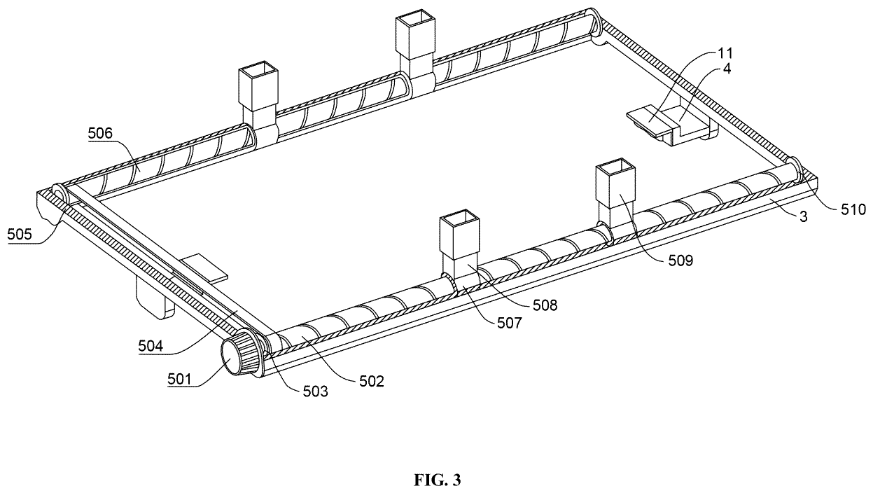

As shown in FIG. 3, a rib-reinforced plate 11 is fixedly connected to one side of the connecting plate 4, and the top of the rib-reinforced plate 11 is fixedly connected to the cardiac surgery bed 1.

It should be noted that a fixed connection between the rib-reinforced plate 11 and the cardiac surgery bed 1 plays a role of reinforcing the connecting strength between the connecting plate and the cardiac surgery bed 1.

As shown in FIG. 2 and FIG. 3, the moving assembly 5 includes a servo motor 501. A left side of the servo motor 501 is fixedly connected to the connecting support shell 3, and an output end of the servo motor 501 runs through an inner cavity of the connecting support shell 3 and is fixedly connected with a first threaded rod 502. A first driving wheel 503 is fixedly connected to one side of a surface of the first threaded rod 502, a surface of the first driving wheel 503 is sleeved with a conveyor belt 504, a front side of an inner cavity of the conveyor belt 504 is in transmission connection with a second driving wheel 505, and a second threaded rod 506 is fixedly connected to an inner cavity of the second driving wheel 505. Both sides of the surface of each of the first threaded rod 502 and the second threaded rod 506 are sleeved with thread bushings 507. An extension plate 508 is fixedly connected to the top of each thread bushing 507, and a limiting shell 509 is fixedly connected to the top of the extension plate 508. Both sides of each of the first threaded rod 502 and the second threaded rod 506 are provided with bearing housings 510, and opposite sides of the two bearing housings 510 are both fixedly connected to an inner wall of the connecting support shell 3.

It should be noted that the output end of the servo motor 501 is driven to make the first threaded rod 502 rotate, the rotation of the first threaded rod 502 drives the first driving wheel 503 to rotate, the rotation of the first driving wheel 503 drives the conveyor belt 504 to drive, then the conveyor belt 504 drives the second driving wheel 505 to rotate to make the second threaded rod 506 to rotate. In this way, the first threaded rod 502 and the second threaded rod 506 both rotate in the inner cavity of the bearing housing 510, making the thread bushing 507 move. When the thread bushing 507 moves, the extension plate 508 is driven to move, and when the extension plate 508 moves, the limiting shell 509 is driven to move, thus adjusting a position of the mapping assembly 6.

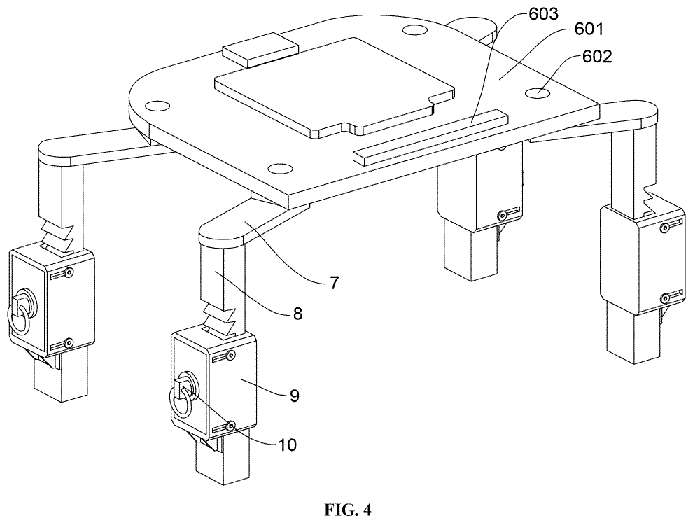

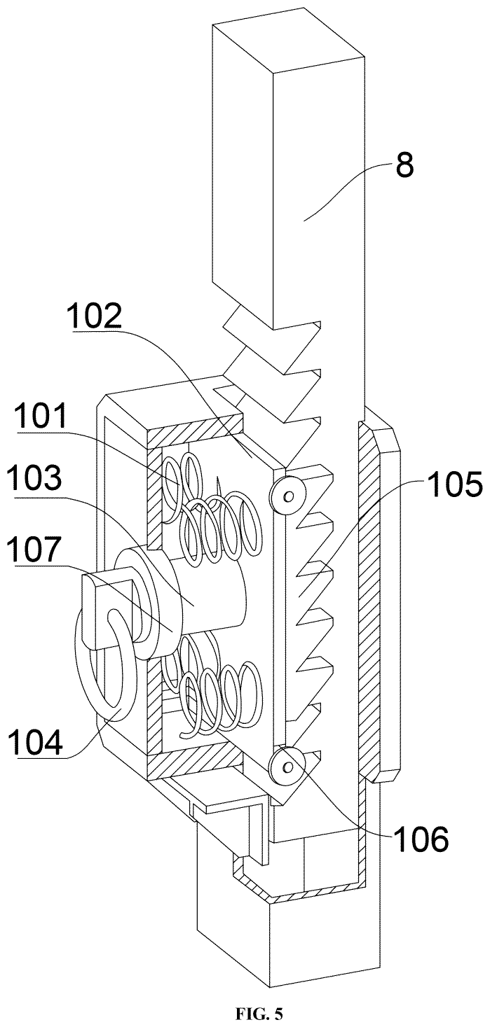

Referring to FIG. 2 and FIG. 4, the mapping assembly 6 includes a protective shell 601, mounting holes 602 are formed in four corners of the top of the protective shell 601, and a hardware assembly 603 is fixedly connected to the top of the protective shell 601. The protective shell 601 is made of PA12, and the hardware assembly 603 includes a magnetic field generator.

It should be noted that the protective shell 601 is convenient for the mounting of the hardware assembly 603. The protective shell is high temperature resistant and corrosion resistant, and thus can effectively protect the mapping assembly 6. The protective shell 601 is made of PA12, which can be disinfected at a high temperature of 120° C., and is more hygienic and safer.

Referring to FIG. 4 and FIG. 5, the adjusting assembly 10 includes a spring set 101. A front side of the spring set 101 is fixedly connected to an inner wall of the positioning shell 9, a positioning plate 102 is fixedly connected to one side of the spring set 101, a connecting rod 103 is fixedly connected to one side of the positioning plate 102, and one side of the connecting rod 103 runs through to the outside of the positioning shell 9 and is fixedly connected with a circular pull ring 104. A tooth block 105 is fixedly connected to one side of the positioning plate 102, and an opening in cooperative use with the tooth block 105 is formed in one side of the guide plate 8. Sliders 106 are fixedly connected to both sides of the positioning plate 102, and opposite sides of the two sliders 106 both run through to the outside of the positioning shell 9. Opening holes in cooperative use with the sliders 106 are formed in both sides of the top and bottom of the inner cavity of the positioning shell 9.

It should be noted that when the user needs to adjust the height of the mapping assembly 6, the circular pull ring 104 can be pulled to make the connecting rod 103 move in an inner cavity of the retaining ring 107 in a limited manner. When the connecting rod 103 moves, the positioning plate 102 is driven to move, when the positioning plate 102 moves, the slider 106 is driven to move in a limited manner to extrude the spring seat 101, and when the positioning plate 102 moves, the tooth block 105 is driven to be separated from the opening on the guide plate 8. In this way, the guide plate 8 can be dragged to drive the mounting plate 7 and the mapping assembly 6 to ascend or descend, then the circular pull ring 104 is released, and the resetting of the spring set 101 enables the positioning plate 102 to drive the tooth block 105 to be inserted into the opening on the guide plate 8, thus fixing the adjusted height.

The working principle is as follows: when a user needs to adjust the mapping assembly 6 to move left and right, an output end of the servo motor 501 is driven to make the first threaded rod 502 rotate, the rotation of the first threaded rod 502 drives the first driving wheel 503 to rotate, the rotation of the first driving wheel 503 drives the conveyor belt 504 to drive, then the conveyor belt 504 drives the second driving wheel 505 to rotate to make the second threaded rod 506 rotate. In this way, the first threaded rod 502 and the second threaded rod 506 both rotate in the inner cavity of the bearing housing 510, making the thread bushing 507 move. When the thread bushing 507 moves, the extension plate 508 is driven to move, and when the extension plate 508 moves, the limiting shell 509 is driven to move, thus adjusting a position of the mapping assembly 6. When the user needs to adjust the height of the mapping assembly 6, the circular pull ring 104 can be pulled to make the connecting rod 103 move in an inner cavity of the retaining ring 107 in a limited manner. When the connecting rod 103 moves, the positioning plate 102 is driven to move, when the positioning plate 102 moves, the slider 106 is driven to move in a limited manner to extrude the spring seat 101, and when the positioning plate 102 moves, the tooth block 105 is driven to be separated from the opening on the guide plate 8. In this way, the guide plate 8 can be dragged to drive the mounting plate 7 and the mapping assembly 6 to ascend or descend, then the circular pull ring 104 is released, and the resetting of the spring set 101 enables the positioning plate 102 to drive the tooth block 105 to be inserted into the opening on the guide plate 8, thus fixing the adjusted height. The protective shell 601 is convenient for the mounting of the hardware assembly 603, can effectively protect the mapping assembly 6 due to its high temperature resistance and corrosion resistance. The protective shell 601 is made of PA12, which can be disinfected at a high temperature of 120° C., and is more hygienic and safer.

It should be noted that relational terms such as first and second herein are only used to distinguish one entity or operation from another entity or operation without necessarily requiring or implying any such actual relationship or order between these entities or operations. Moreover, the terms “comprising”, “including” or any other variation thereof are intended to cover non-exclusive inclusion, so that a process, method, article or equipment including a series of elements includes not only those elements, but also other elements not explicitly listed, or elements inherent to such process, method, article or equipment. Without more restrictions, an element defined by the phrase “including one” does not exclude the existence of other identical elements in the process, method, article or equipment including the element.

Although the embodiments of the present disclosure have been shown and described, those skilled in the art can understand that many changes, modifications, replacements and variations can be made to these embodiments without departing from the principles and purposes of the present disclosure, and the scope of the present disclosure is defined by the claims and their equivalents.

What is claimed is:

1. A support device for a magnetic field generator, comprising a cardiac surgery bed (1), wherein the support device further comprises:
a support column (2) arranged at the bottom of the cardiac surgery bed (1), wherein a connecting support shell (3) is arranged on a surface of the cardiac surgery bed (1), connecting plates (4) are fixedly connected to both sides of the bottom of the connecting support shell (3), and the top of each connecting plate (4) is fixedly connected to the cardiac surgery bed (1);
a moving assembly (5) arranged on one side of the connecting support shell (3), wherein a mapping assembly (6) is arranged at the top of the cardiac surgery bed (1), and provided with mounting plates (7) at four corners of the bottom; and
a guide plate (8) arranged at the bottom of each mounting plate (7), wherein a positioning shell (9) is arranged on a surface of the guide plate (8), and an adjusting assembly (10) is arranged in an inner cavity of the positioning shell (9).

2. The support device for a magnetic field generator according to claim 1, wherein a rib-reinforced plate (11) is fixedly connected to one side of the connecting plate (4), and the top of the rib-reinforced plate (11) is fixedly connected to the cardiac surgery bed (1).

3. The support device for a magnetic field generator according to claim 1, wherein the moving assembly (5) comprises a servo motor (501), a left side of the servo motor (501) is fixedly connected to the connecting support shell (3), and an output end of the servo motor (501) runs through an inner cavity of the connecting support shell (3) and is fixedly connected with a first threaded rod (502); a first driving wheel (503) is fixedly connected to one side of a surface of the first threaded rod (502), a surface of the first driving wheel (503) is sleeved with a conveyor belt (504), a front side of an inner cavity of the conveyor belt (504) is in transmission connection with a second driving wheel (505), and a second threaded rod (506) is fixedly connected to an inner cavity of the second driving wheel (505).

4. The support device for a magnetic field generator according to claim 3, wherein both sides of the surface of each of the first threaded rod (502) and the second threaded rod (506) are sleeved with thread bushings (507), an extension plate (508) is fixedly connected to the top of each thread bushing (507), and a limiting shell (509) is fixedly connected to the top of the extension plate (508).

5. The support device for a magnetic field generator according to claim 3, wherein both sides of each of the first threaded rod (502) and the second threaded rod (506) are provided with bearing housings (510), and opposite sides of the two bearing housings (510) are both fixedly connected to an inner wall of the connecting support shell (3).

6. The support device for a magnetic field generator according to claim 1, wherein the mapping assembly (6) comprises a protective shell (601), mounting holes (602) are formed in four corners of the top of the protective shell (601), and a hardware assembly (603) is fixedly connected to the top of the protective shell (601).

7. The support device for a magnetic field generator according to claim 6, wherein the protective shell is made of PA (polyamide) 12, and the hardware assembly (603) comprises a magnetic field generator.

8. The support device for a magnetic field generator according to claim 1, wherein the adjusting assembly (10) comprises a spring set (101), a front side of the spring set (101) is fixedly connected to an inner wall of the positioning shell (9), a positioning plate (102) is fixedly connected to one side of the spring set (101), a connecting rod (103) is fixedly connected to one side of the positioning plate (102), and one side of the connecting rod (103) runs through to the outside of the positioning shell (9) and is fixedly connected with a circular pull ring (104); and a tooth block (105) is fixedly connected to one side of the positioning plate (102), and an opening in cooperative use with the tooth block (105) is formed in one side of the guide plate (8).

9. The support device for a magnetic field generator according to claim 8, wherein sliders (106) are fixedly connected to both sides of the positioning plate (102), opposite sides of the two sliders (106) both run through to the outside of the positioning shell (9), and opening holes in cooperative use with the sliders (106) are formed in both sides of the top and bottom of the inner cavity of the positioning shell (9).

10. The support device for a magnetic field generator according to claim 8, wherein a surface of the connecting rod (103) is sleeved with a retaining ring (107), and one side, close to the positioning shell (9), of the retaining ring (107) is fixedly connected to the positioning shell (9).
